(19) United States
(12) Patent Application Publication
(10) Pub. No.: US 2021/0010047 A1
(43) Pub. Date: Jan. 14, 2021

(54) METHOD FOR PREPARING ACTIVE PROTEIN HYDROLYSATES BY HYDROLYZING CEREAL PROTEINS WITH MALT TOGETHER WITH PROTEASES

(71) Applicant: Qiqihar University, Heilongjiang (CN)

(72) Inventors: Xiaolan Liu, Heilongjiang (CN); Xiqun Zheng, Heilongjiang (CN); Junzhong Li, Heilongjiang (CN); Mingyang Song, Heilongjiang (CN)

(21) Appl. No.: 16/843,942

(22) Filed: Apr. 9, 2020

(30) Foreign Application Priority Data
Jul. 8, 2019 (CN) 201910609750.1

(51) Int. Cl. C12P 21/06

(52) U.S. Cl. CPC C12P 21/06

(57) ABSTRACT

This disclosure relates to the technological field of nutrient food, particularly relates to a method for preparing active protein hydrolysates by hydrolyzing cereal proteins with malt together with proteases, including the following steps: (1) grinding the malt, obtaining the malt flour; (2) hydrolyzing cereal proteins with the malt flour together with proteases to prepare active protein hydrolysates. The present invention uses the malt flour together with proteases to hydrolyze cereal proteins, which can effectively reduce the consumption of proteases and save the cost, moreover, aminopeptidases and carboxypeptidases in the malt are able to hydrolyze peptide linkages from the ends of proteins, and the bitter hydrophobic amino acids are cut off, thus effectively decreasing the bitterness of the products, on the other hand, which can increase the additional values of the malt, providing its new applications.

### CROSS REFERENCE TO RELATED APPLICATION

This application claims priority to Chinese Patent Application No. 201910609750.1, filed Jul. 8, 2019, the content of which is incorporated herein by reference in its entirety.

### TECHNICAL FIELD

This disclosure relates to the technological field of nutrient food, particularly relates to a method for preparing active protein hydrolysates by hydrolyzing cereal proteins with malt together with proteases.

### BACKGROUND

Cereal protein is a high-quality protein with low price and high nutritive value, also has the characteristics of low fats and low calories. However, cereal proteins contain antinutritional factors, the digestibility of which was low in animals, resulting in a waste of protein resources, and the functional properties of which would change during the production, limiting the wider applications of cereal proteins. Proteins, modified with proteases, would not only enhance the digestibility of proteins, but also improve the processing characteristics of proteins, thus obtaining physiological activities that complete proteins do not have, releasing active peptide fragments, widening the application fields of cereals, and enhancing the economic values of cereal proteins.

After being hydrolyzed with proteases, proteins would produce bitterness, which is mainly due to the exposure of hydrophobic groups after hydrolysis with proteases. The method of removing bitterness include an isolation method and a covering method as well as an enzymatic method, with the enzymatic method having the lowest economic cost, simple in operation and being convenient. In the enzymatic method for removing bitterness, aminopeptidases and carboxypeptidases were mostly used and were two effective proteases.

Barley is one of the first grains planted, the production of which ranks the fourth among grains, and barley is not the staple grain for humans, primarily being used as raw materials for the production of beers. Malts were produced through drying after sprouting of barley, with various enzymes formed during the sprouting of barley, such as amylases, hemi-cellulases and proteases, and allowing the previous existed non-activating enzymes to be activated and increased, for example, during the sprouting, the enzymatic activities of endopeptidases were increased by 5-6 times, the activities of aminopeptidases were increased by 1.5-2.5 times, all of which could play important roles in hydrolyzing cereal proteins.

### SUMMARY

For reducing the consumption of proteases during hydrolyzing cereal proteins with proteases and decreasing the bitterness of hydrolysates, the present invention discloses a method for preparing active protein hydrolysates by hydrolyzing cereal proteins with malt together with proteases, to effectively reduce the consumption of proteases, and effectively decrease the bitterness of hydrolysates, and improve the taste.

For realizing the above objects, the present invention employs the technical solutions below:

A method for preparing active protein hydrolysates by hydrolyzing cereal proteins with malt together with proteases, which is characterized in that: including the following steps:

(1) grinding the malt, obtaining the malt flour;

(2) hydrolyzing cereal proteins with the malt flour together with proteases, to prepare active protein hydrolysates.

According to another aspect, the above step (2) was specifically: formulating cereal protein powders into a suspension of cereal proteins, which was hydrolyzed by adding the malt flour, then hydrolyzed by adding proteases, the hydrosates were dried.

According to another aspect, the substrate concentration of cereal proteins in the above step (2) was 5-15% (w/v).

According to another aspect, the malt flour in the above step (2) represented 10-30% of the total mass of cereal proteins.

According to another aspect, the ratio of proteases to cereal proteins in the above step (2) was 0.5-5.0% (w/w).

According to another aspect, the hydrolysis conditions during the above by adding the malt flour were: the temperature, 45-55° C., the pH, 5.0-6.0, the time, 1-5 h.

According to another aspect, the hydrolysis conditions by the above adding proteases were: the temperature, 40-70° C., the pH, 7.0-10.0, the time, 1-5 h.

According to another aspect, the drying mode in the above step (2) was freeze drying or spray drying.

According to another aspect, the above proteases were one or several kinds of Flavourzyme, Neutral, papain, Alcalase and Protamex.

According to another aspect, the hydrolysis conditions of the above Flavourzyme were: the temperature, 40-60° C.; the pH, 7.0-8.0; the time, 1-5 h. The hydrolysis conditions of Neutral were: the temperature, 40-60° C.; the pH, 7.0-8.5; the time, 1-5 h. The hydrolysis conditions of the papain were: the temperature, 45-70° C.; the pH, 7.0-8.0; the time, 1-5 h. The hydrolysis conditions of Alcalase were: the temperature, 40-60° C.; the pH, 8.0-10.0; the time, 1-5 h. The hydrolysis conditions of Protamex were: the temperature, 45-65° C.; the pH, 7.5-9.5; the time, 1-5 h.

The present invention possesses the following beneficial effects:

(1) The present invention employed the malt flour together with proteases to hydrolyze cereal proteins, which can effectively reduce the consumption of proteases and save the cost, moreover, aminopeptidases and carboxypeptidases in the malt are able to hydrolyze peptide linkages from the end of proteins, resulting in free amino acids, the bitterness of which was lower than that of original bitter peptides, thus effectively decreasing the bitterness of the products, on the other hand, which can also increase the additional values of the malt, providing it new applications;

(2) The present invention employed the malt flour together with proteases to hydrolyze cereal proteins, the various enzymes contained in the malt can partially change the structures of cereal proteins, which was beneficial for the hydrolysis of cereal proteins with proteases, and can effectively enhancing the hydrolysis degree of cereal proteins by proteases;

(3) The method of the invention could improve the functional properties of the prepared active protein hydrolysates in general, such as the solubility, the oil holding capacity, the foamability, the emulsibility, the emulsion stability, etc.;

(4) The active protein hydrolysates prepared with the method of the invention could be used as native antioxidants and antihypertensions, with higher capacities of scavenging —OH, chelating Fe²⁺, scavenging DPPH, in vitro digestibility and ACE inhibitory activities;

(5) The production equipment concerned in the method provided in the present invention was simple with low investment, which is beneficial for the industrial production.

### BRIEF DESCRIPTION OF THE DRAWINGS

The present invention will be further illustrated in conjunction with accompanying diagrams and embodiments below.

FIG. 1 is the histogram illustrating the solubilities of pea protein and the active protein hydrolysates prepared in the embodiments 1-5 and comparative embodiments 1-5 of the present invention;

FIG. 2 is the histogram illustrating the oil holding capacities of pea protein and the active protein hydrolysates prepared in the embodiments 1-5 and comparative embodiments 1-5 of the present invention;

FIG. 3 is the histogram illustrating the foamabilities of pea protein as well as the active protein hydrolysates prepared in the embodiments 1-5 and comparative embodiments 1-5 of the present invention;

FIG. 4 is the histogram illustrating the emulsibilities of pea protein as well as the active protein hydrolysates prepared in the embodiments 1-5 and comparative embodiments 1-5 of the present invention;

FIG. 5 is the histogram illustrating the emulsion stabilities of pea protein as well as the active protein hydrolysates prepared in the embodiments 1-5 and comparative embodiments 1-5 of the present invention.

### DESCRIPTION OF THE EMBODIMENTS

The present invention will now be further illustrated in detail in conjunction with examples.

The Flavourzyme, Neutral, papain, Alcalase and Protamex (enzymes special for vegetable protein hydrolysis) used in the invention were all purchased from Nanning Donghenghuadao Biotechnology Co., Ltd.

A method for preparing active protein hydrolysates by hydrolyzing cereal proteins with malt together with proteases, including the following steps:

(1) grinding the malt, obtaining the malt flour;

(2) hydrolyzing cereal proteins with the malt flour together with proteases to prepare active protein hydrolysates.

In detailed description, hydrolyzing cereal proteins with the malt flour together with proteases can be classified into three groups: (1) hydrolyzing cereal proteins by adding the malt flour and proteases simultaneously; (2) hydrolyzing cereal proteins by adding the malt flour first, and then adding proteases for synergistic hydrolysis; (3) hydrolyzing cereal proteins by adding proteases first, and then adding the malt flour for synergistic hydrolysis.

In the following specific embodiments 1-10 and comparative embodiments 1-5 provided in the present invention below:

The preparation method of the malt flour was: the malt was ground into powders with a grinder for traditional Chinese medicines, which were sieved over a 60-100 mesh screen to give the malt flour, storing in dry conditions.

The hydrolysis method of cereal proteins was: the dry cereal proteins were ground into powders, prepared into a suspension of cereal proteins, which was hydrolyzed by adding the malt flour first, and then hydrolyzed by adding proteases, upon which they were dried.

Wherein, the solvent for preparing the suspension of cereal proteins may be distilled water, a buffer solution or tap water, etc.; and the cereal proteins may be single cereal protein, such as pea protein, corn protein and soybean protein, etc., and also may be complex protein powders.

In detailed description, cereal protein powders can be used directly to prepare the suspension of cereal proteins, or may be pretreated first and then prepared into a suspension of cereal proteins, including pretreatment methods such as cooking and extruding, after cooking, they were dried and then prepared, while after extruding, they were ground into powders and then prepared.

In detailed description, the hydrolysates may be dried directly to obtain the active protein hydrolysates, or may be filtrated and centrifuged, the filtrate or supernatant of which was dried to obtain the active protein hydrolysates, wherein, the drying mode may be freeze drying or spray drying, etc.

### Example 1

(1) First-step hydrolysis of pea protein with the malt: the substrate concentration was 10% (w/v), the additive amount of malt was 30% (w/w), the temperature was 50° C., the pH was at 5.5, the time was 3 h;

(2) Flavourzyme was added into the solution obtained in step (1) for the second-step hydrolysis: the enzyme substrate ratio was 1.25% (w/w), the temperature was 47° C., the pH was at 7.5, reaction for 3 h.

Comparative embodiment 1 was the same as Embodiment 1 basically, differing in that the pea protein was hydrolyzed only with Flavourzyme.

### Example 2

(1) First-step hydrolysis of pea protein with the malt: the substrate concentration was 10% (w/v), the additive amount of malt was 30% (w/w), the temperature was 50° C., the pH was at 5.5, the time was 3 h;

(2) A neutral protease was added into the solution obtained in step (1) for the second-step hydrolysis: the enzyme substrate ratio was 1.00% (w/w), the temperature was 53° C., the pH was at 8.0, reaction for 3 h.

The comparative embodiment 2 was the same as Embodiment 2 basically, differing in that the pea protein was hydrolyzed only with a neutral protease.

### Example 3

(1) First-step hydrolysis of pea protein with the malt: the substrate concentration was 10% (w/v), the additive amount of malt was 30% (w/w), the temperature was 50° C., the pH was at 5.5, the time was 3 h;

(2) A papain was added into the solution obtained in step (1) for the second-step hydrolysis: the enzyme substrate ratio was 1.25% (w/w), the temperature was 50° C., the pH was at 8.0, reaction for 2 h.

The comparative embodiment 3 was the same as Embodiment 3 basically, differing in that the pea protein was hydrolyzed only with a papain.

### Example 4

(1) First-step hydrolysis of pea protein with the malt: the substrate concentration was 10% (w/v), the additive amount of malt was 30% (w/w), the temperature for hydrolysis was 50° C., the pH was at 5.5, the time was 3 h;

(2) An alkaline protease was added into the solution obtained in step (1) for the second-step hydrolysis: the enzyme substrate ratio was 1.25% (w/w), the temperature was 55° C., the pH was at 10.0, reaction for 4 h.

The comparative embodiment 4 was the same as Embodiment 4 basically, differing in that the pea protein was hydrolyzed only with an alkaline protease.

### Example 5

(1) First-step hydrolysis of pea protein with the malt: the substrate concentration was 10% (w/v), the additive amount of malt was 30% (w/w), the temperature for hydrolysis was 50° C., the pH was at 5.5, the time was 3 h;

(2) A protamex protease was added into the solution obtained in step (1) for the second-step hydrolysis: the enzyme substrate ratio was 1.25% (w/w), the temperature was 60° C., the pH was at 9.0, reaction for 4 h.

The comparative embodiment 5 was the same as Embodiment 5 basically, differing in that the pea protein was hydrolyzed only with a protamex protease.

### Example 6

(1) First-step hydrolysis of corn protein with the malt: the substrate concentration was 15% (w/v), the additive amount of malt was 30% (w/w), the temperature for hydrolysis was 55° C., the pH was at 6.0, the time was 5 h;

(2) A Flavourzyme was added into the solution obtained in step (1) for the second-step hydrolysis: the enzyme substrate ratio was 5% (w/w), the temperature was 60° C., the pH was at 7.5, reaction for 5 h.

### Example 7

(1) First-step hydrolysis of corn protein with the malt: the substrate concentration was 5% (w/v), the additive amount of malt was 10% (w/w), the temperature for hydrolysis was 45° C., the pH was at 5.0, the time was 1 h;

(2) A neutral protease was added into the solution obtained in step (1) for the second-step hydrolysis: the enzyme substrate ratio was 1.0% (w/w), the temperature was 40° C., the pH was at 8.0, reaction for 2 h.

### Example 8

(1) First-step hydrolysis of soybean protein with the malt: the substrate concentration 8% (w/v), the additive amount of malt was 15% (w/w), the temperature for hydrolysis was 50° C., the pH was at 5.5, the time was 2 h;

(2) A papain was added into the solution obtained in step (1) for the second-step hydrolysis: the enzyme substrate ratio 0.8% (w/w), the temperature was 70° C., the pH was at 7.0, reaction for 1 h.

### Example 9

(1) First-step hydrolysis of soybean protein with the malt: the substrate concentration was 10% (w/v), the additive amount of malt was 20% (w/w), the temperature for hydrolysis was 50° C., the pH was at 5.5, the time was 3 h;

(2) An alkaline protease was added into the solution obtained in step (1) for the second-step hydrolysis: the enzyme substrate ratio was 0.5% (w/w), the temperature was 50° C., the pH was at 9.0, reaction for 5 h.

### Example 10

(1) First-step hydrolysis of corn protein with the malt: the substrate concentration was 12% (w/v), the additive amount of malt was 25% (w/w), the temperature for hydrolysis was 50° C., the pH was at 5.5, the time was 4 h;

(2) A protamex protease was added into the solution obtained in step (1) for the second-step hydrolysis: the enzyme substrate ratio was 3% (w/w), the temperature was 55° C., the pH was at 8.5, reaction for 3 h.

The active protein hydrolysates prepared in Embodiments 1-5 and Comparative Embodiments 1-5 were prepared into a sample at a concentration of 2 mg/mL for test, meanwhile the un-hydrolyzed pea protein was used as the blank control, with the test results showing in Table 1 (data in the parts with “-” in the table were not given).

TABLE 1

| | | | | | | | | | | | | Pea |
|---|---|---|---|---|---|---|---|---|---|---|---|---|
| | | Embodiments | | | | | Comparative Embodiments | | | | | Pro- |
| | | 1 | 2 | 3 | 4 | 5 | 1 | 2 | 3 | 4 | 5 | tein |
| Solubility (%) | | 85.87 | 93.49 | 93.48 | 100.0 | 94.07 | 70.65 | 73.91 | 54.35 | 100.0 | 85.62 | 56.15 |
| Oil holding | | 4.63 | 3.85 | 5.26 | 5.00 | 2.17 | 7.29 | 7.70 | 9.18 | 3.15 | 2.58 | 1.33 |
| capacity (mL/g) | | | | | | | | | | | | |
| Foamability (%) | | 34.09 | 28.90 | 37.79 | 5.00 | 4.16 | 34.14 | 33.77 | 32.98 | 8.26 | 6.18 | 29.76 |
| Emulsibility | | 14.56 | 14.64 | 37.49 | 2.12 | 15.59 | 7.97 | 6.79 | 7.47 | 8.35 | 12.64 | 32.06 |
| (m²/g) | | | | | | | | | | | | |
| Emulsion | | 56.03 | 58.90 | 74.82 | 43.59 | 46.3 | 62.12 | 71.90 | 73.92 | 86.40 | 64.20 | 61.84 |
| Stability (%) | | | | | | | | | | | | |
| •OH | | 65.42 | 53.10 | 72.28 | 77.02 | 27.37 | 43.83 | 61.79 | 56.08 | 69.42 | 29.41 | 25.69 |
| Scavenging | | | | | | | | | | | | |
| Capacity (%) | | | | | | | | | | | | |
| Fe²⁺ Chelating | | 47.80 | 48.78 | 80.98 | 90.45 | 92.66 | 22.75 | 47.63 | 43.36 | 88.67 | 91.63 | 11.11 |
| Capacity (%) | | | | | | | | | | | | |
| DPPH | | 38.95 | 21.64 | 14.44 | — | — | 23.79 | 18.24 | 19.81 | — | — | 7.02 |
| Scavenging | | | | | | | | | | | | |
| Capacity (%) | | | | | | | | | | | | |
| In | First-step | 0.531 | 0.465 | 0.558 | — | — | 0.368 | 0.379 | 0.310 | — | — | 0.215 |
| vitro | digestion | | | | | | | | | | | |
| Digestibility | (Pepsin) | | | | | | | | | | | |
| | Second-step | 0.432 | 0.338 | 0.714 | — | — | 0.419 | 0.397 | 0.352 | — | — | 0.356 |
| | digestion | | | | | | | | | | | |
| | (Trypsin) | | | | | | | | | | | |

| | | | | | | | | | | | |
|---|---|---|---|---|---|---|---|---|---|---|---|
| ACE inhibitory | 79.45 | — | — | — | — | 53.62 | — | — | — | — | 0.00 |
| activities (%) | | | | | | | | | | | |

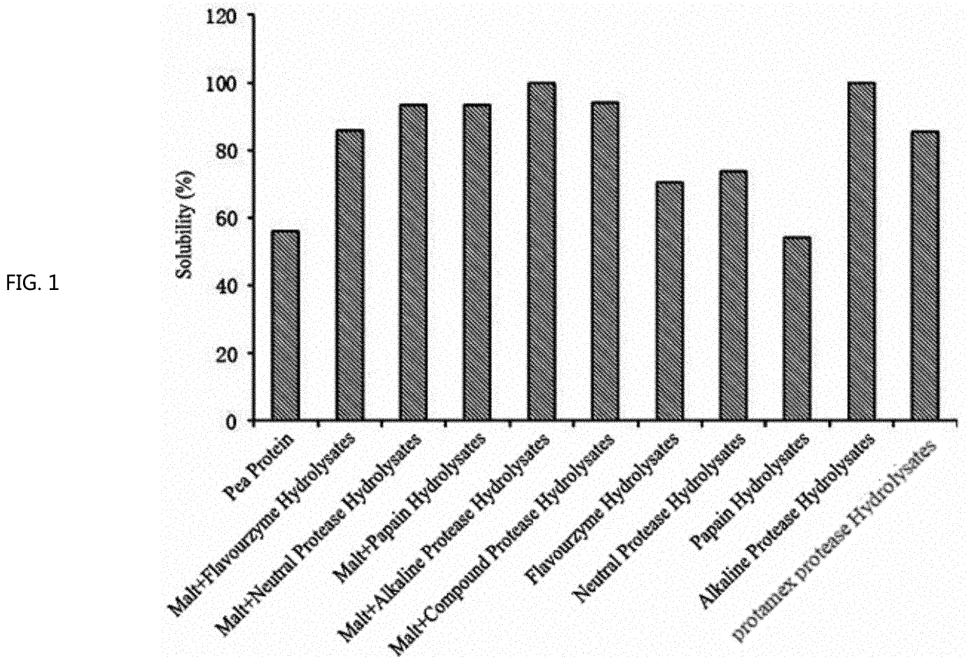

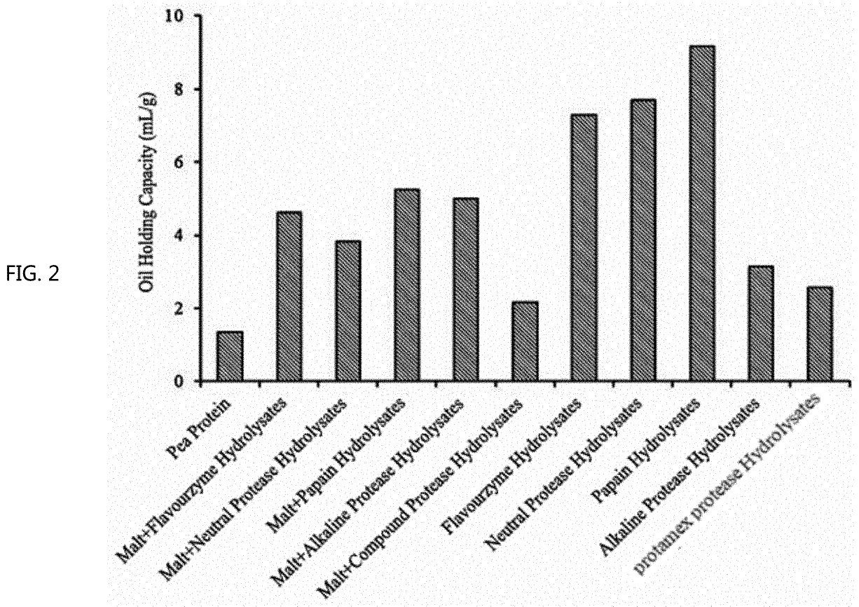

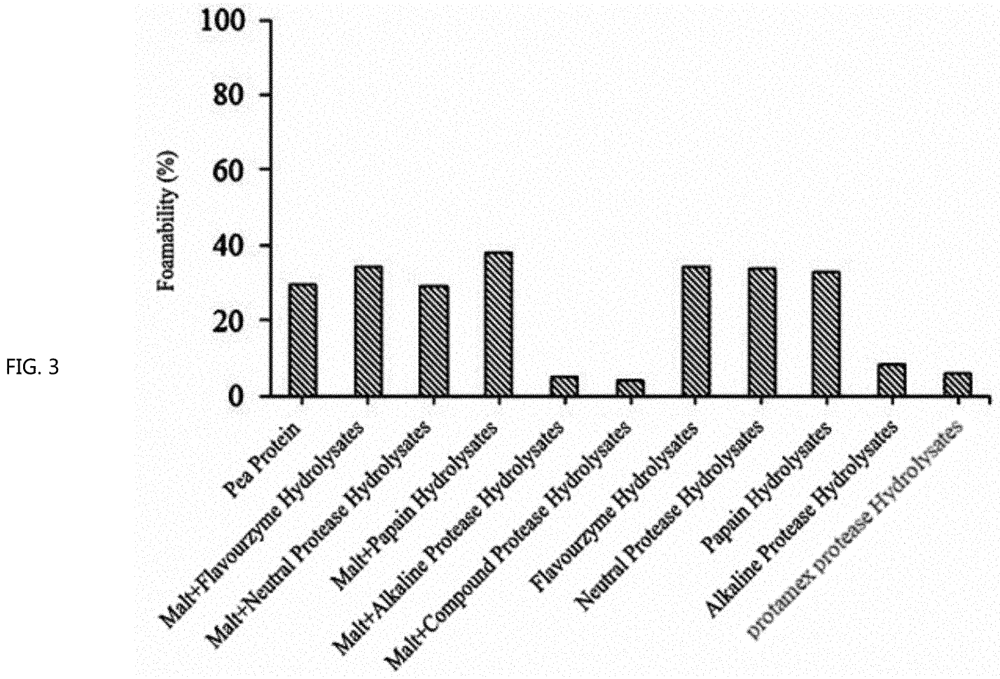

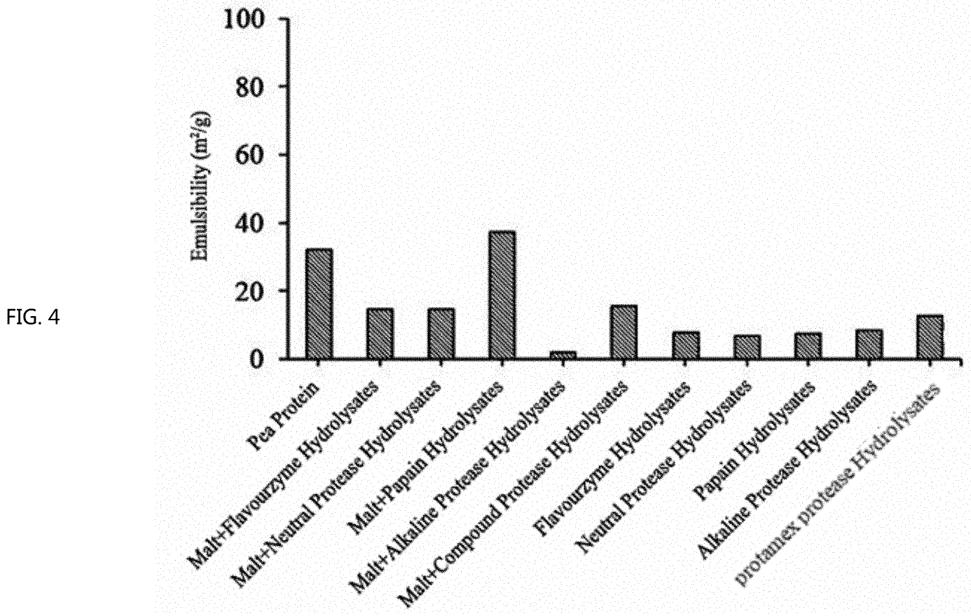

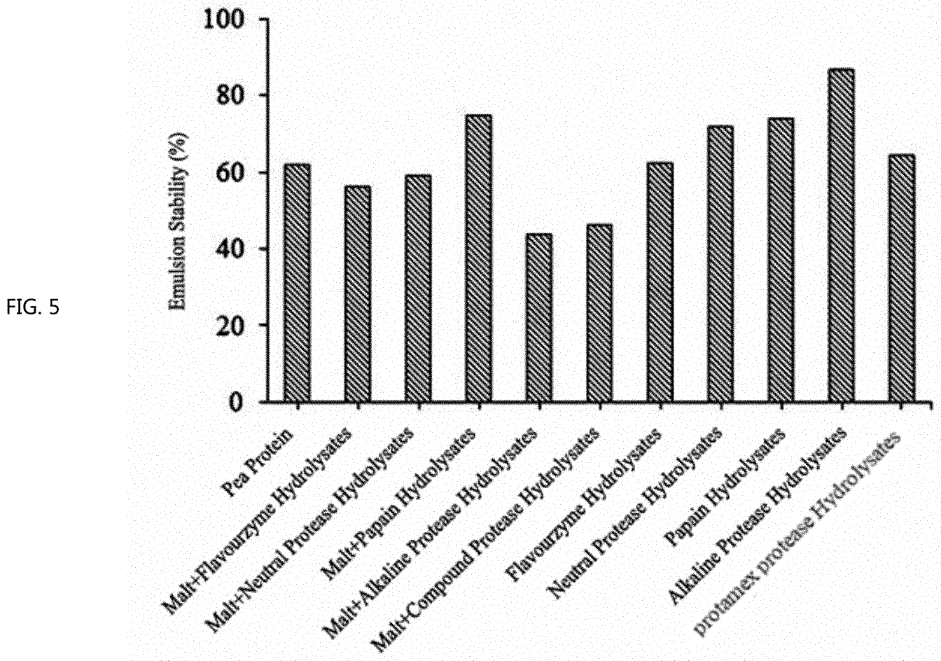

FIGS. 1-5 were the histograms of the solubility, the oil holding capacity, the foamability, the emulsibility and the emulsion stability of pea protein and active protein hydrolysates prepared in Embodiment 1-5 and Comparative Embodiments 1-5 of the invention, respectively.

With the teachings of the above ideal embodiments following the present invention, through the above detailed description, persons with related skills could entirely make various changes and modifications without deviating from the spirit and scope of the present invention. The technical ranges of the present invention were not limited to the contents in the description, which must be defined by the scope of claims.

1. A method for preparing active protein hydrolysates by hydrolyzing cereal proteins with malt together with proteases, which is characterized in that: including the following steps:
(1) grinding the malt, obtaining the malt flour;
(2) hydrolyzing cereal proteins with the malt flour together with proteases to prepare active protein hydrolysates.

2. The method for preparing active protein hydrolysates by hydrolyzing cereal proteins with malt together with proteases of claim 1, which is characterized in that: the step (2) was specifically: grinding dry cereal proteins into powders, which were prepared into a suspension of cereal proteins, hydrolyzed by adding the malt flour first, then hydrolyzed by adding proteases, upon which they were dried.

3. The method for preparing active protein hydrolysates by hydrolyzing cereal proteins with malt together with proteases of claim 2, which is characterized in that: the substrate concentration of cereal proteins in the step (2) was 5-15% (w/v).

4. The method for preparing active protein hydrolysates by hydrolyzing cereal proteins with malt together with proteases of claim 2, which is characterized in that: the malt flour in the step (2) represented 10-30% of the total mass of cereal proteins.

5. The method for preparing active protein hydrolysates by hydrolyzing cereal proteins with malt together with proteases of claim 2, which is characterized in that: the enzyme substrate ratio of proteases to cereal proteins in the step (2) was 0.5-5.0% (w/w).

6. The method for preparing active protein hydrolysates by hydrolyzing cereal proteins with malt together with proteases of claim 2, which is characterized in that: the hydrolysis conditions during the hydrolysis by adding the malt flour were: the temperature, 45-55° C.; the pH, 5.0-6.0; the time, 1-5 h.

7. The method for preparing active protein hydrolysates by hydrolyzing cereal proteins with malt together with proteases of claim 2, which is characterized in that: the hydrolysis conditions during the hydrolysis by adding proteases were: the temperature, 40-70° C.; the pH, 7.0-10.0; the time, 1-5 h.

8. The method for preparing active protein hydrolysates by hydrolyzing cereal proteins with malt together with proteases of claim 2, which is characterized in that: the drying mode in the step (2) was freeze drying or spray drying.

9. The method for preparing active protein hydrolysates by hydrolyzing cereal proteins with malt together with proteases of claim 1, which is characterized in that: the proteases were one or several kinds of Flavourzyme, Neutral, papain, Alcalase and Protamex 8.

10. (canceled)

11. The method for preparing active protein hydrolysates by hydrolyzing cereal proteins with malt together with proteases of claim 2, which is characterized in that: the proteases were one or several kinds of Flavourzyme, Neutral, papain, Alcalase and Protamex.

12. The method for preparing active protein hydrolysates by hydrolyzing cereal proteins with malt together with proteases of claim 3, which is characterized in that: the proteases were one or several kinds of Flavourzyme, Neutral, papain, Alcalase and Protamex.

13. The method for preparing active protein hydrolysates by hydrolyzing cereal proteins with malt together with proteases of claim 4, which is characterized in that: the proteases were one or several kinds of Flavourzyme, Neutral, papain, Alcalase and Protamex.

14. The method for preparing active protein hydrolysates by hydrolyzing cereal proteins with malt together with proteases of claim 5, which is characterized in that: the proteases were one or several kinds of Flavourzyme, Neutral, papain, Alcalase and Protamex.

15. The method for preparing active protein hydrolysates by hydrolyzing cereal proteins with malt together with proteases of claim 6, which is characterized in that: the proteases were one or several kinds of Flavourzyme, Neutral, papain, Alcalase and Protamex.

16. The method for preparing active protein hydrolysates by hydrolyzing cereal proteins with malt together with proteases of claim 7, which is characterized in that: the proteases were one or several kinds of Flavourzyme, Neutral, papain, Alcalase and Protamex.

17. The method for preparing active protein hydrolysates by hydrolyzing cereal proteins with malt together with proteases of claim 8, which is characterized in that: the proteases were one or several kinds of Flavourzyme, Neutral, papain, Alcalase and Protamex.

18. The method for preparing active protein hydrolysates by hydrolyzing cereal proteins with malt together with proteases of claim 10, which is characterized in that: the hydrolysis conditions of the Flavourzyme were: the temperature, 40-60° C.; the pH, 7.0-8.0; the time, 1-5 h. The hydrolysis conditions of Neutral were: the temperature, 40-60° C.; the pH, 7.0-8.5, the time, 1-5 h. The hydrolysis conditions of papain were: the temperature, 45-70° C.; the pH, 7.0-8.0; the time, 1-5 h. The hydrolysis conditions of Alcalase were: the temperature, 40-60° C.; the pH, 8.0-10.0; the time, 1-5 h. The hydrolysis conditions of Protamex were: the temperature, 45-65° C.; the pH, 7.5-9.5; the time, 1-5 h.

19. The method for preparing active protein hydrolysates by hydrolyzing cereal proteins with malt together with proteases of claim 11, which is characterized in that: the hydrolysis conditions of the Flavourzyme were: the temperature, 40-60° C.; the pH, 7.0-8.0; the time, 1-5 h. The hydrolysis conditions of Neutral were: the temperature, 40-60° C.; the pH, 7.0-8.5, the time, 1-5 h. The hydrolysis conditions of papain were: the temperature, 45-70° C.; the pH, 7.0-8.0; the time, 1-5 h. The hydrolysis conditions of Alcalase were: the temperature, 40-60° C.; the pH, 8.0-10.0; the time, 1-5 h. The hydrolysis conditions of Protamex were: the temperature, 45-65° C.; the pH, 7.5-9.5; the time, 1-5 h.

20. The method for preparing active protein hydrolysates by hydrolyzing cereal proteins with malt together with proteases of claim 12, which is characterized in that: the hydrolysis conditions of the Flavourzyme were: the temperature, 40-60° C.; the pH, 7.0-8.0; the time, 1-5 h. The hydrolysis conditions of Neutral were: the temperature, 40-60° C.; the pH, 7.0-8.5, the time, 1-5 h. The hydrolysis conditions of papain were: the temperature, 45-70° C.; the pH, 7.0-8.0; the time, 1-5 h. The hydrolysis conditions of Alcalase were: the temperature, 40-60° C.; the pH, 8.0-10.0; the time, 1-5 h. The hydrolysis conditions of Protamex were: the temperature, 45-65° C.; the pH, 7.5-9.5; the time, 1-5 h.
